(19) United States
(12) Patent Application Publication
(10) Pub. No.: US 2012/0122187 A1
(43) Pub. Date: May 17, 2012

(54) METHOD FOR INCREASING THE CONCENTRATION OF COLONIES OF MICRO ORGANISMS IN A PROCESS FOR REMOVING CONTAMINANTS BY ANAEROBIC DIGESTION

(75) Inventor: Geraldo Nogueira LOPES, JR., Sao Paulo (BR)

(73) Assignee: MERCOSUL COMERCIAL LTDA., Sao Paulo (BR)

(21) Appl. No.: 13/233,487

(22) Filed: Sep. 15, 2011

Related U.S. Application Data
(63) Continuation of application No. 13/020,458, filed on Feb. 3, 2011, now abandoned.
Application No. 13/020,458 is a continuation of application No. 12/810,988, now abandoned.

(30) Foreign Application Priority Data
Dec. 27, 2007 (BR) PI 0705361-4

(51) Int. Cl. C12N 1/20

(52) U.S. Cl. 435/252.1

(57) ABSTRACT

The present invention refers to a process to increase the concentration of micro organisms' colonies which are formed on the surface of Gramineas bambusoideae in a batch and/or continuous flow process which utilizes biomass as a filtration means in order to remove nitrates as well as organic and inorganic impurities from water and/or from affluents in which a step of adsorption is followed by a step of biological degradation by the anaerobic digestion of micro organisms of the types Pseudonomas SP (Nitrosomonas, Nitrosococus, Nitrobacter, Azobacter, Azotomas and Rhixobium). In accordance to the present invention, the addition of about 200-300 ppm of sodium acetate to the solution fed to the reactor, maintaining a relation of 2:1, C:N, provides an efficiency increase of 80% to 98% regarding the removal of nitrate as well as organic and inorganic soluble matter from the water.

This application is a Continuation of U.S. Ser. No. 13/020,458 which is a Continuation of U.S. Ser. No. 12/810,988 filed Jun. 28, 2010, which is a National Stage Application of PCT/BR2008/000404, filed Dec. 26, 2008.

### BACKGROUND OF THE INVENTION

1. Field of the Invention

The present invention refers to a process to increase the concentration of micro organisms colonies formed on the surface of the Gramineas bambusoideae making use of a batch process and/or of continuous flow which utilises biomass as a means for filtration in order to remove nitrates as well as organic and inorganic impurities from water and/or domestic as well as industrial affluent and effluents, in which a step of adsorption is followed by a step of biological degradation by the anaerobic digestion of the micro organisms of the type Pseudonomas SP (Nitrosomonas, Nitrosococus, Nitrobacter, Azobacter, Azotomas and Rhixobium).

2. Description of the State of the Art

As it is well known by the general public, the competitiveness rise in the international market has intensified the importing countries' demands on industrial processes with regards to the assurance not only for the quality of the final product, but also for the preservation of the environment.

This very question resulted on the implementation of quality and environmental certifications such as the ISO 9000 and the ISO 14000, which have both increased the interest of the industries regarding the optimizing of its industrial processes with a particular emphasis towards the environmental question, especially those associated to water.

Although Brazil is one of the countries holding the biggest flows of internal waters, it has been facing a huge disparity regarding the distribution of this natural resource amongst its different geographical regions. To the effect that, as in the North of the Country water abounds, in the South and in the Southeast, industrialized and densely populated areas, due to the high consumption and the unforgiving merciless pollution of the rivers, resulting from a very precarious basic sanitation situation and due to the agricultural fertilization, there is a critic shortage of water.

This very picture causes serious problems to public health and to the environment. Thus, one of the present challenges in order to improve this situation is the development of simple, efficient and adaptable to the economic and structural conditions of the Country, water treatment systems.

Throughout the last decades, in Brazil and all over the world, pollution control has been characterised by a very big effort with regards to the development of carbonated pollution removal technologies. Thus, within the sphere of the gradual rising of demands for pollution control one finds the challenge to find alternatives for the removal of nitrogenous compounds from residual waters from domestic and industrialized affluents and effluents.

In order to ensure a better response towards the environment and towards the society, it is of extreme importance for these water and/or affluents/effluents treatment stations, particularly those based on the implementation of experimental technologies, to be projected and indeed implemented in a very reliable form.

The nitrogen compounds, in its different states of oxidation: ammoniacal and albuminoidal nitrogen, nitrite and nitrate, are amongst the substances which constitute hazard for human health.

Ammonia may be naturally found in superficial as well as subterranean waters, typically in a rather low concentration due to its easy adsorption for soil particles or due to the oxidation to nitrites and nitrates. Nevertheless, the occurrence of higher concentrations can be the result of nearby pollution sources, as well as the reduction of nitrate by bacteria or by ironbound ions found in the soil. The presence of ammonia produces a significant effect in the process of water disinfestations by chlorine through the formation of chloramines, which present a low bactericidal power.

The nitrate is one of the most found ions in natural waters, generally in a very low level in superficial waters, but able to reach very high concentrations in deep waters. Its consumption through the supplied water is associated to two adverse effects to ones health: (i) the inducing to metemoglobulinaemia, especially in children; and (ii) the potential formation of carcinogenic nitrosamines and nitrosamides.

The development of metemoglobulinaemia from the nitrate found in drinking water depends on its bacterial conversion in nitrate during digestion, something which may occur in the saliva and in the gastrointestinal system. Small children, more specifically those younger than 3 months old, are particularly susceptible to the development of this disease due to the more alkaline conditions of their gastrointestinal tract, a factor which is also observed in adult individuals whom suffer from gastroenteritis and anaemia, or those whom have had portions of their stomach surgically removed, as well as in pregnant women.

In Brazil, and in many other countries, the cases of subterranean water contaminated by nitrates are rather frequent, particularly in areas of intensive agricultural activity.

When the concentrations are found to be over and above the Maximum Admissible Amount for waters for human consumption, and unviable to use another supply source, the treatment is essentially indispensable, or else public health will be most definitely at risk.

The typically utilized methods for water treatment with the objective to remove nitrates comprise the steps of adsorption and biological des-nitrification.

One of the important solutions for water des-nitrification for human consumption occurs through an anaerobic digestion, which is the transformation of organic matter in methane and carbon dioxide through a complex macrobiotic system which functions under the lack of oxygen. This very technique consumes a small amount of energy, produces a small amount of slime and generates a usable combustible biogas directly in the production area, and thus it is a method which is more and more applied for des-polluting residual waters.

The des-nitrification itself is the reduction of the nitrates in anoxic conditions, also referred to as dissimulation and biological reduction, in which the bacteria utilizes nitrates, instead of oxygen, as final acceptors of electrons.

This process is characterized by two types of reaction: in the first reaction the nitrate is reduced to nitrite, which is then reduced to gaseous products such as molecular nitrogen or nitrous oxide in a process also referred to as nitrate respiration. The next reaction characterizes the very first step of the des-nitrification process:

NO₃→NO₂→NO→N₃O→N₂

The second reaction involves the nitrate reduction to ammonia via nitrite in a process referred to as ammonification which occurs in conjunction with the process of methane genesis. The electrons donor can be obtained by the addiction of a carbon external source or by the usage of the already existing carbon in the effluent to be treated. The step of des-nitrification is carried out by bacteria, particularly of the genre Pseudomonas.

Others nitrification bacteria are: Nitrosomonas, Nitrosococus, Nitrobacter, Azobacter, Azotomas and Rhizobium. These are heterotrophic anaerobic bacteria which utilize nitrate as an electron acceptor, in the need of some organic material as an electron donor.

NO³⁻+⅚CH₃OH→⅚CO₂+½H₂OO+OH⁻

The des-nitrification presents itself as something very efficient in concentration relations of organic matter in nitrogen at around 5 g. (OCD) g⁻¹ (N—NO₃⁻) (relation 5/1 OCD/.N—NO₃⁻). The relations below these amounts present a reduction regarding the des-nitrification efficiency and the amounts above result in a excessive yielding of ammonia, not eliminating the nitrogen which is present in the effluent in the form of gases.

Other recent studies in literature do show that nitrate can also be removed by the means of the presence of free ammonia in the environment, in accordance to the following reaction:

3NO₃⁻+5NH₄⁺→4N₂+9H₂O+2H⁺

This reaction is possible due to the favourable energetic situation with regards to Gibbs' free energy which is equal to minus 297 KJ/nol. The reaction must be carried out in an environment with pH values above neutrality due to the formation of toxic nitrous oxides to the micro organisms in an acid means.

These very micro organisms can be developed and inoculated utilizing biomass resources, as for instance, the bamboo, which is easily and abundantly found in various regions of the world.

### SUMMARY OF THE INVENTION

The processes which utilize biomass, particularly the bamboo (Gramineas bambusoideae), for the treatment and for the removal of organic and inorganic impurities from water for human consumption and from domestic and industrial effluents, are well known in the state of the art.

However, these rather well known processes in the state of the art present an inconvenience of the necessity of a long time for the concentration of micro organisms' colonies to form on the surface of the Gramineas bambusoideae so that it may reach the minimum level required to ensure an efficient operation of the process.

The objective of the present invention is to put an end to this inconvenience providing a fast quantitative increase on the available organic matter in the means so that the concentration of the micro organisms' colonies to form on the surface of the Gramineas bambusoideae may reach the minimum level required to ensure an efficient operation of the process.

According to the present invention, this objective is reached by the addition of approximately 200-300 ppm of sodium acetate to the fed solution to the reactor thus keeping the rate of 2:1 C:N, which accelerates the growth of the said colonies.

### DESCRIPTION OF THE INVENTION

In order to develop a natural treatment process that enables the reduction of nitrate as well as organic and inorganic impurities content in contaminated subterranean waters and/or domestic and industrial affluents and effluents, the viability and the operational conditions of a physic and chemical nitrate adsorption process were studied, followed by the des nitrification of biological digestion. For this very purpose, piston flux bamboo reactors with the activated slime adsorbed by the internal wall and by the bamboo surface were utilized. The same was used as a filtration means.

For the adsorption step, the fundamental parameter for the unit project in real scale is the loading that measures the amount of contaminants removed by mass unit of adsorbent. This very result informs the saturation time of a determined column and the necessary mass of filtration means for the removal of the contaminants, in this case, nitrate.

### Removal of Nitrate by Physic—Chemical Adsorption and Biological Degradation

Two types of adsorbents were used to measure the efficiency of the nitrate removal en natural waters through the adsorption process: activated coal and bamboo.

The activated coal, supplied by from the company Carbonifera Catarinense S/A was milled until it reached a particle diameter compatible to the sieve's mesh of 80 and 100 size.

The bamboo was utilized in two formats. First they were prepared in disks with a medium mass of 25 g and following that, the milled bamboo was obtained with a compatible particle dimension to the sieve's mesh of 30-100 size, it was cleaned with a solution of NaOH 0.1 M for the removal of soluble in water compounds and it was dried in a stove at 105° C. for two hours.

The water utilized in the tests was simulated through the use of distilled water with the addition of a amount of sodium nitrate sufficient enough to simulate concentrations of 10 to 500 mg/L of N—NO₃.

The adsorption assays were conducted in a process of batch regime as well as in a process of continuous flow. In each batch 1000 mL of water were added containing a nitrate concentration (N—NO₃) of 10 to 500 mg/L. The systems were kept under constant stirring (100 rpm) at room temperature (20 to 40° C.), in a pH 3-9.

The adsorption capacity was determined through the measure of remaining nitrate concentration in the solution after the adsorption step by the method described under the Norm NBR 12620/92-Nitrate determination-chromo topic acid and phenol disulfonic acid methods.

Mathematically, the adsorption capacity is expressed in relation to the loading of nitrate under the surface of the adsorbent through the following material balance:

Mass_{adsorbed}=Mass_{removed}

q=[(Cₒ−C_{f})·V]/W

where Cₒ and C_{f} represent the nitrate concentrations before and after the adsorption, respectively, V is the solution's volume and W is the absorbent's mass.

### Removal of Nitrate by Biochemical Degradation and Filtration

The bamboo was also utilised here in order to promote the nitrate's removal by biological digestion, by bio degradation of organic and inorganic compounds found in water e/or domestic and industrial affluents and effluents, specially the nitrate.

In order to carry out this experiment, a solution with an approximate concentration of 20 ppm of NO₃ was prepared. The means to support the generation of micro organisms was bamboo utilized in the experiment immediately after it had been collected. Four reactors were prepared varying the mass of the bamboo in relation to the total volume of the nitrate solution as mentioned in the table 1 below.

The reactors consisted of thermal plastic boxes with an approximate capacity of 225 l of water.

Three reactors were utilized. In each one of them, 8 kg of cut bamboo (transversally cut, in pieces of 30 cm) were added and 80 l of water were supplied by the CASAN—Companhia de Águas e Saneamento do Estado de Santa Catarina (The State of Santa Catarina Company of Water Supply and Sanitation), at the region of Laguna—State of Santa Catarina, with sufficient enough sodium nitrate in order to generate a concentration of 30 ppm of N—NO₃⁻.

TABLE 1
Loading of the biological reactors utilized for the
removal of nitrate

| | | | Proportion (%) |
|---|---|---|---|
| | Bamboo Mass | Solution Volume | m_{bamboo}/ |
| Reactor | (Kg) | (L) | vol_{effluent} |
| 1 | 0.4 | 80 | 0.5 |
| 2 | 0.8 | 80 | 1.0 |
| 3 | 4 | 80 | 5.0 |
| 4 | 8 | 80 | 10.0 |

Sodium nitrate and potassium solutions were prepared sufficient enough to generate a nitrate concentration varying between 10 to 500 mg/L containing bamboo masses in different percentages (1% up to 80%) in relation to the amount of water to be treated.

These very reactors were kept at rest and samples were removed in time intervals varying from 1 to 72 hours. After the end of the process the remaining nitrate concentration was evaluated. After the nitrate's biological degradation, the samples were purified in fast gravity filters.

The filtration means was composed of milled bamboo, sand and activated coal with heights for a capacity of a hydraulic application rate of approximately 200-300 m³/m²·dia⁻¹. The objective of the filtration was to remove suspended particles present in the water resulting from the biological process and to reduce the amount of organic matter acquired in the biological reactor during the biodegradation process.

The evaluation of the filtration efficiency was determined through the content measure of organic matter dissolved in water (ODQ) in the sample obtained from the reactor and in the sample obtained from the water generated by the filter.

These very experiments were kinetically accompanied with the objective of measuring the following transformations:
The reduction of the nitrate concentration with regards to the anaerobic respiration of the micro organisms which utilizes it as the final electrons acceptor for the respiration; and
The parameters alteration in natural water: OCD (Oxygen Chemical Demand), OBD (Oxygen Biochemical Demand), total nitrogen, colour, turbid ness and total suspended solids with regards to the solubility and/or the excretion of metabolites resulting from the microbial activity.

The nitrate, nitrogen, colour, turbidity and total suspended solids analysis were carried out in a Merck® photometer Spectroquant Nova 40 model, in accordance to ISO's recommendations. The OCD and OBD analysis were carried out according to the method described in Standards Methods for the Examination of the Water and Wastewater (APHA, 1995).

The effluents generated by the biological reactors were purified in fast gravity filters. As well as the purification through filtration, the effluents were also oxidized utilizing as oxidizing agent in a concentration equal to 0.5-1.0 ppm. The disinfection has a contact time of 20 minutes. After these two operations the effluents had the OCD, OBD, colour, turbid ness and total suspended solids parameters determined.

The effluents generated by the biological reactors were purified in fast gravity filters containing the following composition of filtration means presented on Table 2.

TABLE 2
Composition of the filtration means utilized for the
purification of the effluents generated by the biological reactor

| Material | Height of the means (m) |
|---|---|
| Sand | 0.15 |
| Activated Coal | 0.35 |
| Pebbles | 0.15 |

As well as the purification through filtration, the effluents were also oxidized utilizing sodium hypochlorite as an oxidizing agent in a concentration equal to 0.5-1.0 ppm. The disinfection has a contact time of 20 minutes. After these two operations the effluents had the OCD, OBD, colour, turbid ness and total suspended solids parameters determined.

TABLE 3
The limits for the analysed parameters

| | Maximum Permitted | |
|---|---|---|
| Parameter | Value (MPV) | Reference |
| Colour (Hz) | 15 | Official Notice No. |
| | | 518 of the MS |
| Turbid ness (NTU) | 5 | Official Notice No. |
| | | 518 of the MS |
| Total Suspended | none | Official Notice No. |
| Solids (mg/L) | | 518 of the MS |
| OBD₅ (mg/L¹) | 3 | CONAMA (*) |
| | | Resolution No. 357 - |
| | | Fresh waters of |
| | | Class 1 |
| NO₃⁻—N (mg/L) | 10 | Official Notice No. |
| | | 518 of the MS |

(*) CONAMA = Conselho Nacional de Meio Ambiente (The National Council for the Environment).

With the view to increase the amount of organic matter available in the means, in accordance to the present invention, sodium acetate was added to the solution fed to the reactors 5 and 6, with a difference that in the reactor 6 the pH of the means was closed off with NaHCO₃ in order to exclude any interference of acidity of the means in the activity of the micro organisms going through a process of des-nitrification.

According a second well know art literature, the best condition for the occurrence of a biological digestion in a environment catalyzed by micro organisms is when the relation carbon:nitrogen (C:N) is two to 1 (2:1).

Thus, considering the carbon present in the acetate and nitrogen in the nitrate, the stechiometric balance of the reaction 1 indicates the necessity for the addition of approximately 204 ppm of acetate for the amount of nitrate which was simulated in the experiments (please refer to the Tables).

The experiments carried out with molar relation above 2:1 (C:N) demonstrated that the reactions kinetics of biological degradation is somewhat favourable, at least until a molar relation of approximately 350 ppm. Above this very amount, the experiments demonstrated that when the acetate concentration in water is rather increased, problems start to appear such as the excess of organic matter in water by the end of the process.

On the other hand, the experiments with a variation of the amount of acetate with amounts below 220 ppm demonstrated that the biological digestion is slower, thus not being favourable to degradation.

The best kinetics values were obtained with molar relations between 200 and 300 ppm, in other words, with these concentrations we were able to obtain the best times of hydraulic detention for the reactions.

A third reactor 7 was utilized as a blank test in order to compare the influence of the acetate and the bicarbonate addition in the des-nitrification process. The composition of the reactors 5, 6 and 7 is shown on Table 4.

TABLE 4
Loading of the biological reactors utilized

| | | Solution | | | |
|---|---|---|---|---|---|
| | | Volume | Proportion | | Concen- |
| | Bamboo | (L) | (%) | Concentration | tration |
| Reactor | Mass | 30 ppm | M_{bamboo}/ | NaAc | NaHCO₃ |
| No. | (kg) | N—NO₃⁻ | Vol_{effluent} | (ppm) | (ppm) |
| 5 | 8 | 80 | 10 | 204 | — |
| 6 | 8 | 80 | 10 | 204 | 324 |
| 7 | 8 | 80 | 10 | — | — |

### Results and Arguments

The results obtained in the above mentioned experiments can be observed in the following Table 5:

TABLE 5
Quality of the water obtained after the treatment in the reactors 5, 6 and 7

| | Reactor 5 | | | Reactor 6 | | | Reactor 7 | | |
|---|---|---|---|---|---|---|---|---|---|
| Time (h) | 0 | 20 | 40 | 0 | 20 | 40 | 0 | 20 | 40 |
| NO₃⁻ | 139.2 | 128.9 | 72.8 | 131.2 | 130.8 | 114 | 129.7 | 126.8 | 110 |
| (mg · L⁻¹) | | | | | | | | | |
| N— NO₃⁻ | 31.6 | 29.3 | 16.5 | 29.8 | 29.7 | 25.9 | 29.5 | 28.8 | 25.0 |
| (mgN · L⁻¹) | | | | | | | | | |
| Colour (Hz) | 21.4 | 35.9 | 50.6 | 0.7 | 12.6 | 27.1 | 4.0 | 13.1 | 23 |
| Turbidness (NTU) | 7 | 11 | 17 | 1 | 4 | 10 | 4.0 | 5 | 8 |
| SST (ppm) | 2 | 4 | 10 | 0 | 12 | 7 | 0 | 0 | 0 |

The reactors 5 and 6 had the bamboos utilized in the first reaction with 40 hours, applied again in a new reaction cycle. The objective of this study was to evaluate if the adaptation phase of the micro organism to the environment can be accelerated if one uses bamboo with a microbial activity already developed. The composition of the reactor 8 and 9 are shown on Table 6.

TABLE 6
Loading of the biological reactors utilized for the
removal of nitrate with the addition of nutrients

| | Reactor 8 | | | Reactor 9 | | |
|---|---|---|---|---|---|---|
| Time (h) | 0 | 20 | 40 | 0 | 20 | 40 |
| NO₃⁻ | 135.9 | 65.3 | 0.6 | 133.9 | 86.9 | 3.3 |
| (mg · L⁻¹) | | | | | | |
| N— NO₃⁻ | 30.9 | 14.8 | 0.13 | 30.4 | 19.7 | 0.75 |
| (mgN · L⁻¹) | | | | | | |
| Colour (Hz) | 15.1 | 61.3 | 119.9 | 1.1 | 47 | 88.9 |
| Turbidness (NTU) | 5 | 24 | 58 | 2.0 | 24 | 48 |
| SST (ppm) | 5 | 16 | 48 | 0 | 21 | 44 |

By looking at the results we may observe that the addition of sodium acetate favours the des-nitrification reaction if we compare the results obtained by the reactor 5 in relation to the reactor 7 which had no addition of acetate.

After 40 hours of reaction time it is possible to reduce the nitrate concentration (N—NO₃⁻) in reactor 5 from 31.6 down to 16.5 ppm achieving a reduction of approximately 48% whilst the reactor 7 only reduces the nitrate concentration (N—NO₃⁻) from 29.5 down to 25 ppm thus representing a reduction of about 13% in the same time interval.

The addition of sodium bicarbonate (reactor 6) interferes with the kinetics of the reaction at the first utilization of the bamboo in relation to the observed kinetics in the reaction without the bicarbonate (reactor 5), the reduction on the concentration of nitrate in reactor 6 being somewhat similar to that observed in reactor 7. When the bamboo is utilized in a new experiment, we can observe that the kinetics of des-nitrification é favoured.

In the reactor 5 the final concentration of nitrate is 0.13 ppm and the des-nitrification efficiency is around 99.5%.

In the second utilization of the bamboo in the reactor which received an addition of bicarbonate, we can observe little difference when compared to the reactor 5, but the removal of nitrate efficiency in the reactor 5 in the second utilization was of approximately 97.5%.

Thus we can conclude that the addition of acetate in the means does accelerate the des-nitrification measured by the micro organisms generated by the bamboo.

Apart from this very fact, the efficiency of the process reaches values of around 99.5% for the bamboo utilized for the second time, probably because of the fact that the microorganisms have already been developed on the bamboo which is being utilized for the second time. One experiment was carried out utilizing the bamboo of reactor 5 for the third time. The results are demonstrated below in Table 7.

TABLE 7
Results obtained with the reaction carried out in
reactor 5 after the third utilization of the bamboo
Reactor 5

| | Time (h) | | |
|---|---|---|---|
| | 0 | 20 | 40 |
| NO₃⁻ | 135.9 | 70 | 0.7 |
| (mg · L⁻¹) | | | |
| N—NO₃⁻ | 30.9 | 15.9 | 0.7 |
| (mgN · L⁻¹) | | | |
| Colour (Hz) | 13.9 | 50.3 | 110.6 |
| Turbid ness | 3 | 21 | 58 |
| (NTU) | | | |
| SST (ppm) | 0 | 14 | 54 |

By looking at the above demonstrated results it is possible to verify that the results obtained in the second utilization of the bamboo are very similar indeed to the ones observed in the third utilization of the bamboo in reactor 5.

As and when the des-nitrification is complete, the characteristics of the water do alter, particularly the colour which actually reaches values above 100 Hz.

Also, the turbid ness and the concentration of suspended solids increase with the des-nitrification. The sample obtained after the reaction in reactor 5, once the des-nitrification is complete, was utilized so that we may verify the efficiency of the filter described in the section previous to the conditioning of the water for drinking purposes. The results are shown in table 9.

TABLE 9
Results obtained with the filtration of effluents
generated by the reactor 5 in its second utilization after the
passage by the sand, coal and pebbles filter.

Reactor 5

| | Entering the | Exiting the |
|---|---|---|
| Parameter | filter | filter |
| Colour (Hz) | 119.5 | 24.5 |
| Turbid ness | 58 | 21 |
| SST (ppm) | 48 | 0 |

From the data above mentioned it is possible to observe that the filter applied for the water purification obtained from the reactor 5 promotes the reduction of the parameters considered for this study. Nevertheless, the values are situated above the demanded limits for drinking water which means that new studies must be carried out to increase the filter efficiency and/or new processes such as flocculation and oxidative may be considered.
The results obtained with the process in accordance to the present invention demonstrated that the amount of bamboo, in other words, its percentage in volume in relation to the amount of water or effluent to be treated does significantly influence the quality of the water obtained after the process, regardless if the process is by adsorption or biological degradation.

1. A process to increase the concentration of micro organisms colonies in a process for the removal of impurities by anaerobic digestion in a reactor which utilizes the micro organisms colonies formed in the Gramineas bambusoideae as a filtration means, characterized by the fact that it comprises the addition of an amount of sodium acetate stechiometrically acceptable to the solution fed to the reactor.

2. A process to increase the concentration of micro organisms colonies in a process for the removal of impurities by anaerobic digestion according to claim 1, characterized by the fact that the said stechiometrically acceptable amount varies between approximately 200-300 ppm of sodium acetate.

3. A process to increase the concentration of micro organisms colonies in a process for the removal of impurities by anaerobic digestion according to claim 2, characterized by the fact that the said stechiometrically acceptable amount maintains a molar rate of 2:1 of C:N.

4. A process to increase the concentration of micro organisms colonies in a process for the removal of impurities by anaerobic digestion according to claim 3, characterized by the fact that the said reactor is a piston flux reactor.
